(19) United States
(12) Patent Application Publication
(10) Pub. No.: US 2007/0293942 A1
(43) Pub. Date: Dec. 20, 2007

(54) PROSTHETIC VALVE AND DEPLOYMENT METHOD

(76) Inventor: Daryush Mirzaee, Sunnyvale, CA (US)

Correspondence Address: HENRICKS SLAVIN AND HOLMES LLP;SUITE 200, 840 APOLLO STREET, EL SEGUNDO, CA, 90245, US

(21) Appl. No.: 11/424,690

(22) Filed: Jun. 16, 2006

(51) Int. Cl. A61F 2/24; A61F 2/06

(52) U.S. Cl. 623/2.11; 623/2.18; 623/1.24

(57) ABSTRACT

Prosthetic valves that are adapted to be expanded into deployment and shrunk for repositioning and methods that are applicable to such valves.

### BACKGROUND OF THE INVENTIONS

Valve replacement is sometimes necessary in those instances where a patient experiences heart valve stenosis or regurgitation. Prosthetic valves typically include two structures, a leaflet device that consists of one or more leaflets which perform the opening and closing functions of the replaced biological valve and an anchor that holds the leaflet device in place.

Valve replacement was for many years a highly invasive open heart procedure. During open heart surgery, the patient is placed under general anesthesia and connected to a heart-lung bypass machine so that blood can continue to circulate during the procedure. Access to the heart is obtained by way of a sternotomy. The defective valves were typically excised and prosthetic valves were implanted in their place. Although such procedures represented an advance in the area of heart valve stenosis and regurgitation treatment, there are a number of risks associated with open heart valve replacement procedures. Some risks, such as adverse reactions to the anesthesia, bleeding, and infections, are associated with surgical procedures in general. Other risks, such as death, stroke, heart attack, arrhythmia, and kidney failure, are more closely associated with open heart surgery. Surgical valve replacement may also be painful and require prolonged hosptialization.

More recently, percutaneous heart valve replacement has been proposed as a less invasive alternative to open heart valve replacement procedures. Percutaneous valve replacement procedures often involve delivering a collapsed prosthetic valve to the deployment location (e.g. the mitral valve or aortic valve) on the distal end of a catheter. Once the prosthetic valve has reached the deployment location, the valve is deployed by expanding the anchor into contact with tissue in such a manner that the valve will not move.

Percutaneous heart valve replacement has proven to be a significant advance because it eliminates many of the risks and other shortcomings associated with open heart valve replacement procedures. Nevertheless, the present inventor has determined that percutaneous heart valves, and the associated methods of deployment, are susceptibe to improvement. For example, the present inventor has determined that it can be quite difficult to move conventional prosthetic valves after they have been deployed in those instances where the deployment location is determined to be suboptimal. A subobtimal deployment location may, for example, be the result of less than optimal initial deployment of the valve or an anatomic shift that could occur years after a sucessful initial deployment.

### SUMMARY OF THE INVENTIONS

A prosthetic valve in accordance with one embodiment of a present invention includes an anchor that is configured to be expanded to a deployment size during the deployment process and to shrink to a smaller repositioning size when exposed to a condition that is not a normal body condition. A method in accordance with one embodiment of a present invention includes the step of shrinking a prosthetic valve by causing the valve anchor to transition from the martensitic state to the austenitic state.

The present apparatus and methods provide a number of advantages over conventional apparatus and methods. For example, the present apparatus and methods allow valves that are at a less than optimal location to be simply and easily disengaged from the associated tissue structure (e.g. the tissue associated with the mitral valve or aortic valve), moved to a more optimal location and redeployed. Alternatively, if necessary, the disengaged valve may be percutaneously withdrawn from the patient. The present apparatus and methods are also less complicated than conventional apparatus and methods. As a result, the present apparatus, as compared to conventional valves, is easier to make and use, is less expensive, and may be deployed with a smaller delivery system to better facilitate percutaneous delivery. The present apparatus may also be deployed with a balloon or other inflatable structure, which physicians tend to be comfortable with.

The above described and many other features and attendant advantages of the present inventions will become apparent as the inventions become better understood by reference to the following detailed description when considered in conjunction with the accompanying drawings.

### BRIEF DESCRIPTION OF THE DRAWINGS

Detailed description of preferred embodiments of the inventions will be made with reference to the accompanying drawings.

FIG. 1 is a side view of a prosthetic valve in accordance with one embodiment of a present invention.

FIG. 2 is a front view of the prosthetic valve illustrated in FIG. 1.

FIG. 3 is a side view of a prosthetic valve anchor in accordance with one embodiment of a present invention.

FIG. 4 is a plan view of a prosthetic valve and a delivery system in accordance with one embodiment of a present invention.

FIG. 5 is a side view of a prosthetic valve and an expandable device in accordance with one embodiment of a present invention.

FIG. 6 is a section view taken along line 6-6 in FIG. 4.

FIGS. 7A-7D are partial section views showing a prosthetic valve being deployed in accordance with one embodiment of present invention.

FIGS. 8A-8F are partial section views showing a prosthetic valve being repositioned and redeployed in accordance with one embodiment of present invention.

FIG. 9 is a side view of a prosthetic valve anchor in accordance with one embodiment of a present invention.

FIG. 10 is a front view of a prosthetic valve anchor in accordance with one embodiment of a present invention.

FIG. 11 is a side view of a prosthetic valve anchor in accordance with one embodiment of a present invention.

FIG. 12 is a side, section view of a prosthetic valve anchor in accordance with one embodiment of a present invention.

FIG. 13 is a side, section view of a prosthetic valve anchor in accordance with one embodiment of a present invention.

FIG. 14 is a side, section view of a prosthetic valve anchor in accordance with one embodiment of a present invention.

FIG. 15A is a side view of a prosthetic valve and an expandable device in accordance with one embodiment of a present invention.

FIG. 16A is a side view of a prosthetic valve on an unexpanded expandable device in accordance with one embodiment of a present invention.

FIG. 16B is a side view of a prosthetic valve on an expanded expandable device in accordance with one embodiment of a present invention.

FIG. 16C is a side view of an expanded expandable device in accordance with one embodiment of a present invention.

FIG. 16D is a side view of a prosthetic valve on an expanded expandable device in accordance with one embodiment of a present invention.

FIG. 17 is a side view of an expandable device in accordance with one embodiment of a present invention.

FIG. 18 is a side, cutaway view of an expandable device in accordance with one embodiment of a present invention.

FIG. 19 is a side, cutaway view of an expandable device in accordance with one embodiment of a present invention.

FIG. 20 is a side view of a prosthetic valve anchor in accordance with one embodiment of a present invention.

FIG. 21 is a partial section view showing a prosthetic valve and delivery system in accordance with one embodiment of present invention.

### DETAILED DESCRIPTION OF THE PREFERRED EMBODIMENTS

The following is a detailed description of the best presently known modes of carrying out the inventions. This description is not to be taken in a limiting sense, but is made merely for the purpose of illustrating the general principles of the inventions. Additionally, although the present inventions are discussed below in the context of heart valves, the inventions herein also have application in other regions of the body such as, for example, the esophagus, stomach, ureter, vesica, biliary passages, lymphatic system, intestines, and veins outside the heart.

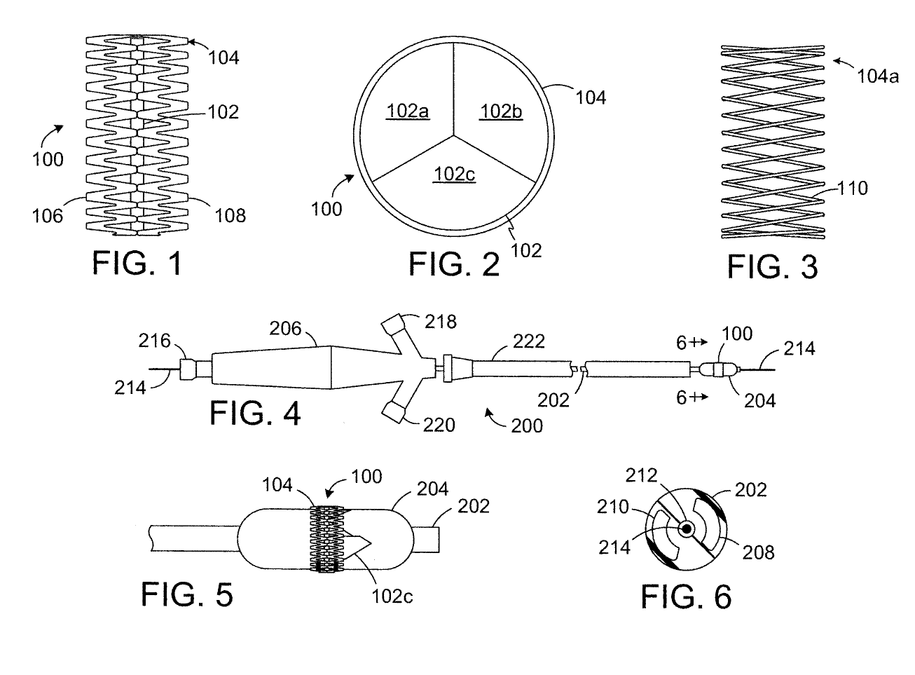

As illustrated for example in FIGS. 1 and 2, a prosthetic valve 100 in accordance with one embodiment of a present invention includes a leaflet device 102 and an anchor 104. The leaflet device 102 is configured to allow flow in one direction in response to a pressure differential across the prosthetic valve 100 and to prevent flow when the pressure differential is reversed. As such, the prosthetic valve 100 is well-suited for applications within the heart and may be used to replace one or more of the aortic, mitral, tricuspid and pulmonary valves. Although the present inventions are not limited to any particular leaflet device configuration, the exemplary leaflet device 102 includes three leaflets 102a-c. The leaflets 102a-c may be in the form of biologic tissue leaflets (e.g. cadaver, bovine or porcine tissue) or synthetic leaflets (e.g. metal, polymer or engineered tissue). The leaflets 102a-c may be attached to the anchor 104 by, for example, welding, adhesive bonding, fusing, suturing, stapling, or some combination thereof.

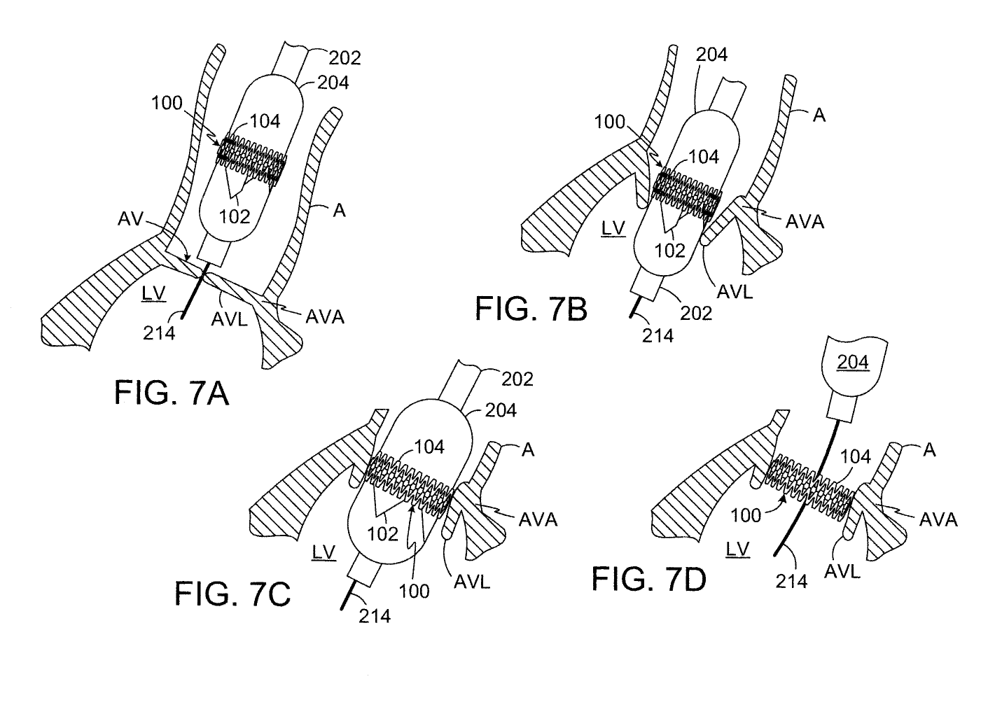

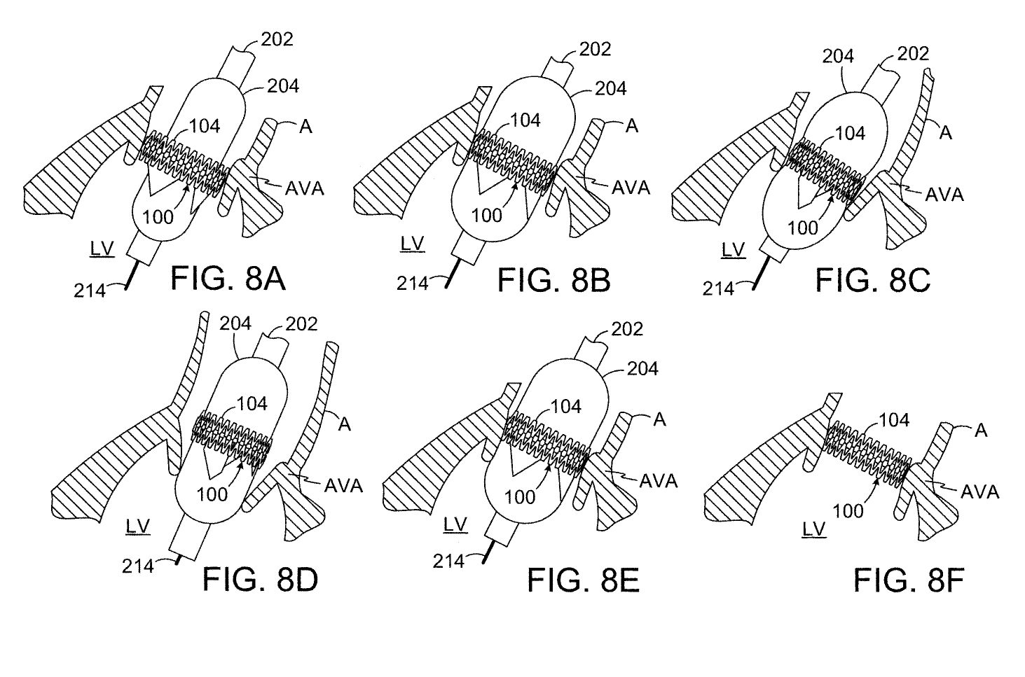

Turning to the exemplary anchor 104, and as discussed in greater detail below in the context of FIGS. 7A-7D and 8A-8F, the anchor is configured such that it may be delivered to a target location at a relatively small delivery size, mechanically deformed to a larger deployment size and, if necessary, activated in such a manner that it will shrink to a repositioning size that is smaller than the deployment size. The exemplary anchor 104 is also configured to remain at the repositioning size after actuation and will not expand back to the deployment size until it is again mechanically deformed. As a result, in those instances where a deployment location is suboptimal, the size of the anchor 104 can be reduced to a point where the prosthetic valve 100 can be disengaged from the surrounding tissue, repositioned and redeployed. In some implementations, the repositioning size will be small enough to facilitate percutaneous withdrawal of the valve from the patient, if necessary, in addition to repositioning of the valve for redeployment.

The delivery, deployment and repositioning sizes will depend on the delivery method and the size of bodily region in which the valve is intended to be deployed. In the exemplary context of a prosthetic aortic valve that is delivered percutaneously, the delivery diameter of the exemplary anchor 104 may range from about 4 mm to about 6 mm, the deployment diameter of the anchor may range from about 24 mm to about 30 mm, and the repositioning diameter of the anchor may be about 1 mm or more smaller than the deployed diameter. For example, the repositioning diameter of the anchor 104 may range from slightly smaller than the delivery diameter to at least 1 mm smaller than the deployed diameter. The length of the anchor 104, when expanded to the deployed diameter, may range from about 7 mm to about 20 mm, or longer if the anchor is intended to extend into the aorta and/or left ventricle. One example of a relatively long (i.e. longer than 20 mm) anchor is discussed below with reference to FIG. 15.

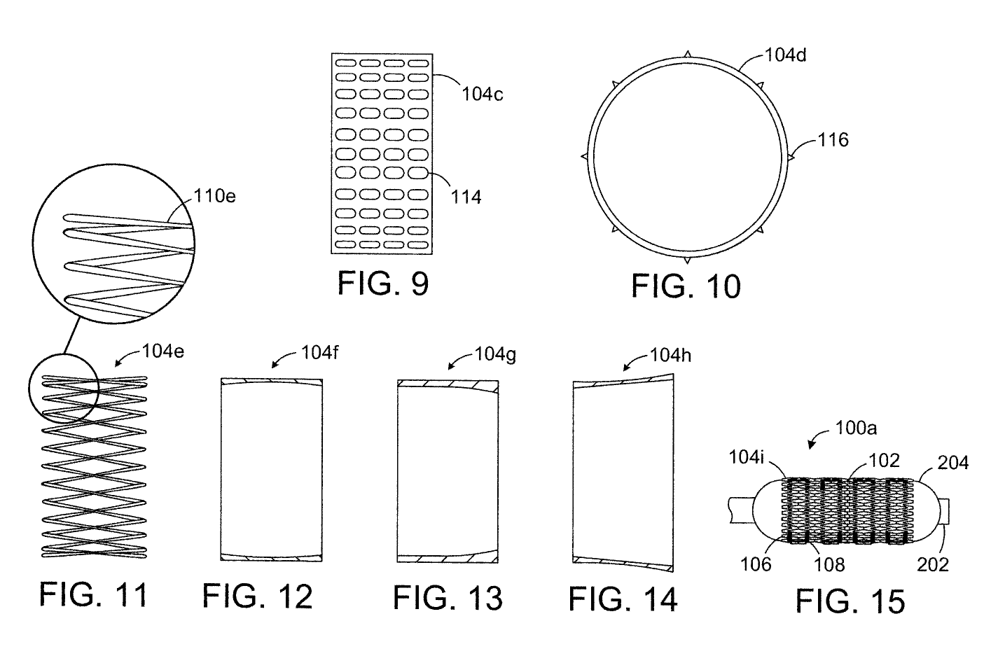

The ability to function in the intended manner at the intended location within the body notwithstanding, the anchor is not limited to any particular mechanical structure. The exemplary anchor 104 illustrated in FIGS. 1 and 2, which is intended for use within the heart, is in the form of a tubular wire mesh. More specifically, the exemplary anchor 104 includes first and second wires 106 and 108 that are angled back and forth and configured into an overall circular shape. The wires 106 and 108 are also secured to one another by, for example welding, bonding or adhesive, to form the tubular structure. Wire mesh structures may be configured in a variety of other ways. By way of example, but not limitation, an anchor may include one or more additional tubular wire structures 106 (or 108). There may also be some overlap of the wire structures 106 and 108, if desired, in order to increase the stiffness of certain portions of the anchor. The exemplary anchor 104a illustrated in FIG. 3 includes one or more wires 110 with portions that are transverse to one another and secured to one another. A single wire wound into a coil is another suitable anchor structure. The anchor 104 may also be formed from a tube with a solid wall or from a perforated sheet that is rolled into a tube, as is discussed below in the context of FIG. 9. It should also be noted that although the exemplary anchor members 104 and 104a, are radially and longitudinally symetrical, they may also be radially and/or longitudinally assymetrical as necessary or desired. This aspect of the present inventions is discussed below in the context of FIGS. 11-14.

With respect to materials, the present anchors are preferably formed from shape memory material that responds to a transition condition that is not a normal body condition. Thermally responsive shape memory materials, which change shape when heated to a predetermined temperature, are one example of a shape memory material that may be employed. Suitable materials include thermally responsive nickel-titanium alloys (e.g. the nickel-titanium alloy sold under the trade name NITINOL), copper-zinc-aluminum alloys, copper-aluminum-nickel alloys and polymers, each with a transition temperature that is slightly higher than the highest expected temperature within the body, i.e. a temperature that is not a normal body condition. The transition temperature should not, however, be high enough to cause appreciable tissue damage after short term exposure. A transition temperature of about 45° C. to 60° C. is suitable given the normal body temperature of 37° C. Alternatively, ferromagnetic shape memory materials, which change shape in response to the application of a magnetic field, may be employed. Given the fact that the body does not generate internal magnetic fields, a magnetic field is a transition condition that is not considered to be a normal body condition.

Regardless of the material chosen for the anchor, the material must be “trained” to function in the manner described above. For example, an anchor formed NITINOL or some other thermally responsive shape memory material may be trained in the following manner. An anchor, such as one of the exemplary anchors 104 and 104a, is initially constructed in any size other than the repositioning size. The anchor is then mechanically deformed to the repositioning size and heat treated to at least the transition temperature, e.g. heated to a temperature of at least about 45° C. to 60° C. in the case of NITINOL, to complete the training. As a result, when the anchor material is in the martensitic state and deformed, such as when the anchor is expanded from the delivery size to the deployment size at body temperature, it will remain deformed when the force responsible for the deformation is discontinued. Such deformation is referred to herein as “mechanical deformation.” However, when the anchor is heated to the transition temperature, it will transition to the austenitic state and return to the repositioning size to which it has been trained. The anchor will also remain in the repositioning size (i.e. the trained size) after cooling to body temperature and returning to the martensitic state. The anchor will only return to the larger deployment size if a deformation force is applied thereto. Ferromagnetic shape memory materials may be trained in a similar manner, albeit one that employs magnetic fields in place of heat.

With respect to delivery and deployment, the exemplary prosthetic valve 100 may be delivered to the aortic, mitral, tricuspid and pulmonary valves, or any other target location, and deployed with any suitable device. One example of such a device is the catheter generally represented by reference numeral 200 and illustrated in FIGS. 4-6. The catheter 200 includes an elongate catheter body 202 with a balloon 204 (or other expandable device) on the catheter body distal end and a handle 206 on the catheter body proximal end. The balloon 204 may be used to hold the prosthetic valve 100 during delivery and also used to deploy and redeploy the valve. The catheter body 202 has infusion and ventilation lumens 208 and 210 that open into the interior of the balloon 204, as well as a central guidewire lumen 212 for a guidewire 214. In the illustrated embodiment, the guidewire lumen 212 extends to the proximal end of the catheter body 202 and the handle 206 includes a guidewire port 216. Alternatively, the guidewire lumen 212 may extend only a few centimeters proximal of the balloon 204 and create an opening on the exterior of the catheter body 202 at that point.

The handle 206 in the exemplary catheter 100 also includes infusion and ventilation ports 218 and 220 that are connected to the catheter body infusion and ventilation lumens 208 and 210. A fluid source (not shown) may be connected to the infusion and ventilation ports 216 and 218 and used to inflate the balloon 204 during deployment of the prosthetic valve 100. The fluid source may also be used to circulate fluid heated to the transition temperature during repositioning. A sheath 222, which may be positioned over the prosthetic valve 100 during delivery in order to prevent the anchor 104 from damaging non-target tissue and/or unintended expansion of the anchor, may also be provided. The sheath 222 may be moved proximally from its position over the prosthetic valve 100 just prior to reaching the target location, or after reaching the target location but prior to deployment.

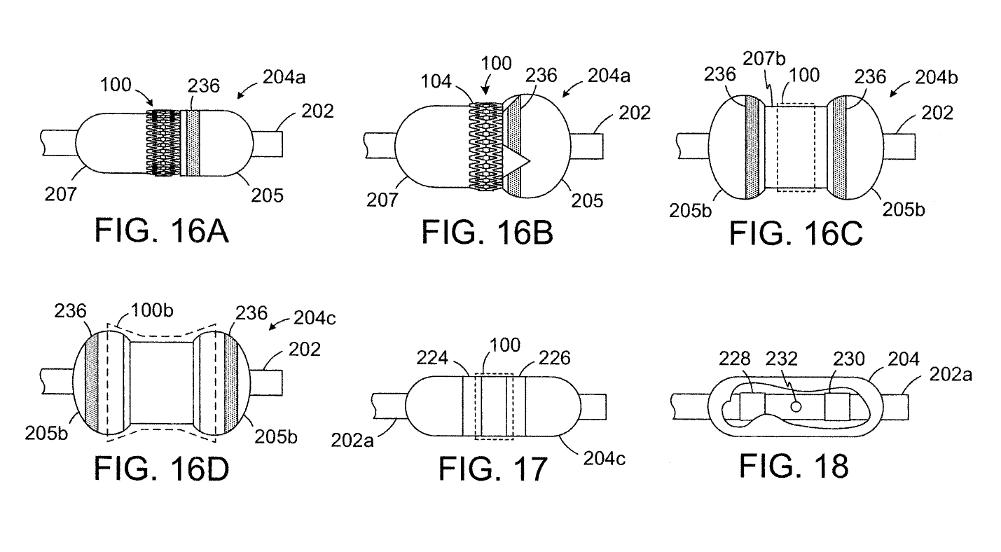

With respect to dimensions and material, the exemplary catheter body 202 will typically be about 5 mm in diameter and may be formed from any suitable biocompatible material. Such materials include, for example, biocompatible thermoplastic materials such as Pebax® material, polyethylene, or polyurethane. The balloon 204 will preferably be formed from material that is relatively high in thermal conductivity. Suitable materials for the balloon include thermally conductive biocompatible materials such as silicone, polyisoprene, Nylon, Pebax®, polyethylene, polyester and polyurethane. The uninflated and fully inflated sizes of balloon 204 will depend on the delivery and deployment sizes of the prosthetic valve with which it is intended to be used. The balloon 204 may also be provided with radiopaque markers (discussed below in the context of FIGS. 16A-16C), either on the balloon itself or on portions of the catheter body 202 within the balloon. Such markers help the physician align the balloon with the valve 100.

Referring to FIG. 5, the exemplary prosthetic valve 100 will typically be crimped onto the balloon 204 prior to the start of the delivery and deployment procedure. More specifically, the prosthetic valve 100 will be placed, while at the repositioning size, over the uninflated balloon 204. The valve 100 will then be compressed, such that the anchor 104 is mechanically deformed radially inwardly down to the delivery size, and secured to the uninflated balloon 204 by mechanical interference and friction, as is illustrated in FIG. 5.

As noted above, the exemplary prosthetic valve 100 may be used, in the context of the heart, to replace one or more of the aortic, mitral, tricuspid and pulmonary valves. An aortic valve replacement is shown in FIGS. 7A-7D for illustrative purposes only. Flouroscopy may be used to monitor the position of the prosthetic valve 100 during the procedure. Referring first to FIG. 7A, the guidewire 214 may be advanced into the left ventrical LV by way of the aorta A using a retrograde approach. Antegrade and hybrid approaches may also be employed. The catheter body 202 may then be adavanced over the guidewire 214 with the prosthetic valve 100 crimped onto the balloon 204 and the sheath 222 (FIG. 4) over the prosthetic vavle. The sheath 222 may be withdrawn once the prosthetic valve 100 and balloon 204 are adjacent to the aortic valve AV, as is shown in FIG. 7A. Turning to FIG. 7B, the catheter body 202 may then be advanced until the prosthetic valve 100 and balloon 204 displace the aortic valve leaflets AVL and the prosthetic valve is aligned with the aortic valve annulus AVA. The balloon 204 may then be inflated, as illustrated in FIG. 7C, by filling the balloon with fluid that is below the transition temperature of the thermally responsive shape memory anchor material. The anchor 104, which has remained in the relatively small delivery size up to this point, will be expanded to the deployment size by the balloon 204. The anchor 104 will also be mechanically deformed by the expansion and, accordingly, will remain at the deployment size after the balloon 204 has been deflated and withdrawn from the aortic valve annulus AVA, as illustrated in FIG. 7D. Withdrawal of the balloon 204 will also allow the leaflet device 102 to return to its normally closed orientation.

The same process would be employed in those instances where the anchor is formed from a ferromagnetic shape memory material. Here, however, the temperature of the fluid used to inflate the balloon 204 will not effect the anchor.

It should be noted here that the aortic valve leaflets AVL are displaced toward the left ventricle LV during the delivery and deployment procedure illustrated in FIGS. 7A-7D. This may be reversed, i.e. the aortic valve leaflets AVL may be displaced toward the aorta A, in other procedures that involve the exemplary prosthetic valve 100.

In either case, there will invariably be some instances where the initial deployment location of the prosthetic valve 100 is suboptimal. For example, the location illustrated in FIG. 7D could be deemed to be too close to the left ventricle LV. There will also be some instances where the initial deployment location is optimal and a subsequent situation arises (e.g. an anatomic shift) that results in a suboptimal valve location. Such subsequent situations could arise soon after the catheter is withdrawn, or at a later time, up to many years after the initial deployment. The present prosthetic valve 100, which may be disengaged from the aortic valve annulus AVA, repositioned and redeployed in the exemplary manner illustrated in FIGS. 8A-8F, is especially useful in these situations.

At the outset of the disengagement portion of the repositioning procedure, the catheter body 202 may be moved distally until the balloon 204 is again aligned with the prosthetic valve 100, as illustrated in FIG. 8A. There may also be some instances where the catheter 200 will simply remain in the location illustrated in FIG. 7C (and 8A) until the accuracy of the initial deployment has been evaluated. Turning to FIG. 8B, the balloon 204 may then be inflated into contact with the valve anchor 104 with fluid heated to a temperature above the transition temperature of the material used to form the anchor (about 45° C. to 60° C. in the exemplary embodiment). Preferably, the relatively high temperature fluid will be continuously infused into the balloon 204, and ventilated from the balloon, in order to offset the loss of heat to the surrounding blood that is normally only about 37° C. The heat from the fluid will warm the anchor 104 to at least the transition temperature of the material (e.g. NITINOL) from which the anchor is formed, typically in just a few seconds. In response to being heated to the transition temperature, and as illustrated in FIG. 8C, the anchor material will transition to the austenitic state and the anchor 104 will shrink down to the repositioning size to which it was trained. Shrinkage of the anchor 104 may also cause some deformation of the balloon 204. At this point, the anchor 104 will be disengaged from the aortic valve annulus AVA. In other words, although a portion of the anchor 104 may be in contact with tissue depending on the position of the catheter body 202, the anchor will be readily movable relative to the aortic valve annulus AVA.

The temperature of the fluid circulating through the balloon 204 may, at this point, be reduced to a temperature below the transition temperature (e.g. body temperature or room temperature), thereby returning the thermally responsive shape memory anchor material to the martensitic state. The anchor 104 will remain at the repositioning size after cooling to the temperature below the transition temperature. As illustrated for example in FIG. 8D, the volume of fluid within the balloon 204 may, if desired, also be slightly reduced to the point where the balloon is only as big as is necessary to hold the prosthetic valve 100. The prosthetic valve 100 may then be moved with the catheter 200 to the more optimal location illustrated in FIG. 8D. The balloon 204 may then be inflated, as illustrated in FIG. 8E, by filling the balloon with fluid that is below the transition temperature of the anchor material. The anchor 104, which has remained in the repositioning size up to this point, will again be expanded to the deployment size. The anchor 104 will also be mechanically deformed by the expansion and, accordingly, will remain in the deployment size after the balloon 204 has been deflated and withdrawn from the aortic valve annulus AVA, as illustrated in FIG. 8F. Withdrawal of the balloon 204 will also allow the leaflet device 102 to return to its normally closed orientation.

A substantially similar procedure would be employed in those instances where the anchor is formed from a ferromagnetic shape memory material and FIGS. 8A-8F may be used to desribe such a procedure. In the case of ferromagnetic shape memory material, however, the temperature of the fluid used to inflate the balloon (note FIGS. 8B and 8E) will not effect the anchor. Instead, the anchor will be subjected to a magnetic field that will actuate the ferromagnetic shape memory material when the anchor is to be shrunk from the deployment size down to the repositioning size (note FIG. 8C). The magnetic field will be removed after the anchor reaches the repositioning size and the anchor will remain in the repositioning size until it is again mechanically deformed by the balloon (FIG. 8E).

Although the present inventions have been described in terms of the preferred embodiments above, numerous modifications and/or additions to the above-described preferred embodiments would be readily apparent to one skilled in the art.

By way of example, and as illustrated in FIG. 9, an exemplary anchor 104c is formed from a sheet of shape memory material with a plurality of perforations 114. The perforations may be any suitable shape or size or any suitable combination of different shapes and/or sizes. The exemplary anchor 104d illustrated in FIG. 10 is identical to the anchor 104 but for the inclusion of protrusions 116 that are used to further secure the anchor to the target tissue region after deployment. Other surface structures, such as hooks or surface irregularities, may also be employed.

Turning to FIGS. 11-14, anchors in accordance with the present inventions may also be configured such that the size or geometry of the structures that define the anchors are something other than constant. Referring first to FIG. 11, the exemplary anchor 104e includes a plurality of wires 110e that are transverse to one another and secured to one another at their longitudinal ends. The cross-sectional size of the wires 110e (i.e. diameter in the case of a circular wire) may vary over the length of the wires. In the illustrated embodiment, the cross-sectional size is smallest at the longitudinal mid-point (or some other point between the longitudinal ends), and increases from there to each of the longitudinal ends, where the cross-sectional size is largest. This may also be reversed, such that the cross-sectional size of the wires is largest at the mid-point or some other point between the longitudinal ends. The wire cross-sectional size may, alternatively, be smallest at one longitudinal end, largest at the other, and tapered therebetween. There may also be regions of constant cross-sectional size combined with regions of varying cross-sectional size as well as instances where some wires in an anchor are configured differently than others.

The tubular anchor 104f illustrated in FIG. 12 has a constant outer diameter and an inner diameter that varies such that the wall thickness of the tube is greatest at the longitudinal ends. This may also be reversed so that the wall thickness is greatest at the mid-point or some other point between the longitudinal ends. Alternatively, as illustrated in FIG. 13, the wall thickness is constant over a portion of the length of the anchor 104g and variable over another. This may consist of a constant wall thickness from one longitudinal end to the mid-point, or some other point between the longitudinal ends, and an increasing wall thickness from this point to the other longitudinal end (as shown), a decreasing wall thickness from the this point to the other longitudinal end, or any other combination of constant and non-constant wall thicknesses. As illustrated for example in FIG. 14, the anchor 104h has a non-constant wall thickness as well as a non-constant overall cross-sectional size. More specifically, the overall diameter of the anchor 104h increases from one longitudinal end to the other. Other types of variations in the overall cross-sectional size are contemplated. For example, the overall diameter of the anchor 104h could, alternatively, be greatest at the longitudinal ends whether or not the wall thickness is constant. The tubular anchors illustrated in FIGS. 12-14 may also include apertures, such as those discussed above with reference to FIG. 9, or slots.

An exemplary valve 100a with a relatively long anchor 104i is illustrated in FIG. 15. The anchor 104i includes a plurality of tubular wire structures 106 and 108 secured to one another by, for example welding, bonding or adhesive. The relatively long anchor 104i will typically be longer than 20 mm and may be used, for example, to secure the valve 100a to tissue within the aorta and/or left ventricle.

Alternative balloon structures are also contemplated. The exemplary balloon 204a, which is illustrated in its uninflated state in FIG. 16A and its inflated state in FIG. 16B, includes a distal portion 205 that is slightly larger than the proximal portion 207. The larger distal portion 205 prevents distal movement of the valve 100 after the valve has been crimped onto the uninflated balloon 204a during delivery and recovery of the valve. When the balloon 204a is inflated, it prevents the valve 100 from sliding distally off the catheter 200, especially as the anchor 104 shrinks from the deployed size to the repositioning size. It should be noted that the difference in cross-sectional size (i.e. diameter) is somewhat exaggerated in FIGS. 16A and 16B. In practice, the difference in diameter will be such that the deflated diameter of the balloon distal portion 205 will be small enough to pass through the valve 100 after the anchor has been expanded to the deployment size. Typically, the distal portion 205 will be about 1 mm to about 3 mm larger in diameter than the proximal portion 207 when the balloon 204 is inflated. Turning to FIG. 16C, the exemplary balloon 204b includes proximal and distal portions 205b that are slightly larger than the portion 207b therebetween.

The exemplary balloons 204a and 204b also include radiopaque markers 236. In the illustrated embodiments, the markers 236 are provided on the larger balloon portions 205 and 205b. Alternatively, or in addition, radiopaque markers may be provided on the smaller balloon portions 207 and 207b. Radiopaque markers may, alternatively, be carried by the catheter body 202 within the balloons at locations aligned with those discussed above and/or on other portions of the catheter body.

The balloons 204a and 204b may also be used to carry and deploy a valve that includes an anchor with an overall diameter that, when deployed, is greatest at one or both of the longitudinal ends. As illustrated for example in FIG. 16D, a valve 100b that has been delivered in a crimped state, where the diameter of the anchor is essentially constant over its length, may be expanded during deployment by a balloon 204c in such a manner that the diameter of the anchor is greatest at the longitudinal ends. The balloon 204c is identical to balloon 204b but for the locations of the markers 236.

The manner in which the balloon heats the anchor 104 may also be varied. As illustrated for example in FIG. 17, the balloon 204d includes conductive regions 224 and 226 that may be used to conduct current through the anchor 104 and resistively heat the anchor above the transition temperature. The use of resistive heating obviates the need for heated fluid and the circulation thereof within the balloon. Accordingly, the catheter body 202a includes a single fluid lumen that may be used to both infuse and ventilate fluid in and out of the balloon 204d. Another heating apparatus that does not involve direct heating of the fluid is one or more resistive heaters carried on the exterior or interior of the balloon wall or between the plies of material used to form the balloon.

Another alternative is to heat the fluid while it is in the balloon. To that end, and as illustrated for example in FIG. 18, a pair of electrodes 228 and 230 may be mounted on the catheter body 202a within the balloon 200. The single fluid lumen, which terminates at an aperture 232, may be used to inflate the balloon with a conductive fluid such as saline. Current will be transmitted from one electrode to the other in order to resistively heat the conductive fluid. The single fluid lumen may also be used to ventilate fluid from the balloon 200 when necessary.

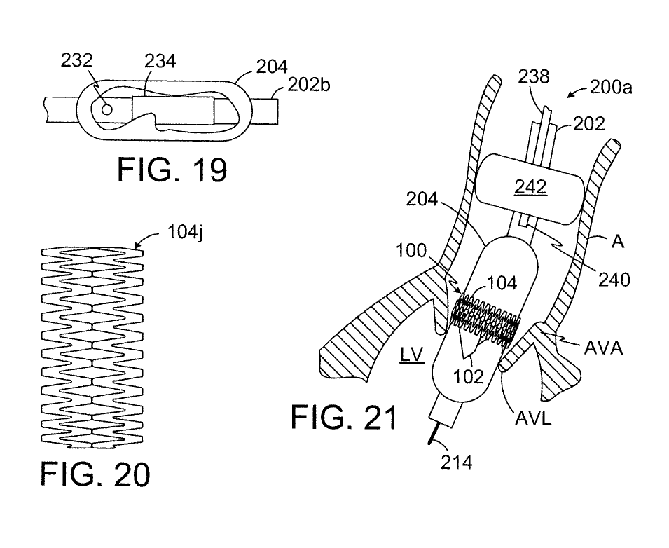

Still another exemplary catheter configuration is illustrated in FIG. 19. The catheter illustrated in FIG. 19, which is intended for use with anchors such as the exemplary anchor 104j illustrated in FIG. 20 that are formed from ferromagnetic shape memory materials, includes a selectively actuatable magnet 234 in place of the electrodes 228 and 230. The magnet 234 may be used to generate a magnetic field sufficient to cause the associated anchor to shrink from the deployment size to the repositioning size. It should be noted here that anchors formed from ferromagnetic shape memory materials may also have any of the other configurations discussed above.

Anchors in accordance with the present inventions may also be heated with fluid, at the appropriate heating temperature, that is simply supplied to the bodily region where the valve is deployed. Suitable fluids include saline and contrast fluid. The catheter 200a illustrated in FIG. 21, which is substantially similar to the catheter 200, is one example of a device that can heat the valve anchor in this manner. The catheter 200a includes a catheter body 202a with a single infusion and ventilation lumen that terminates within the balloon 204. A fluid tube 238, which has an outlet 240 near the proximal end of the balloon 204, is carried on the exterior of the catheter body 202a. Alternatively, the catheter body could be provided with an internal heated fluid lumen that has an outlet at a location similar to that of the outlet 240. In either case, the catheter 200a may also be provided with an occlusion balloon 242 which, in combination with the balloon 204, will prevent blood from cooling the heated fluid being supplied to the anchor 104. In still other implementations, the fluid tube 238 (or internal heated fluid lumen) may be omitted and the heated fluid simply supplied by way of the space between the outer surface of the catheter body 202a and in inner surface of the delivery sheath 222 (FIG. 1).

Prosthetic valves in accordance with the present invention may also include a coating over the anchor, or at least over the anchor surfaces that will be in contact with tissue, that prevents anchor/tissue adhesion. Adhesion prevention facilitates valve repositioning that may be required long after the initial deployment. Polymeric coatings may, for example, be employed for this purpose. Coatings including anti-thrombotic drugs and/or other therapeutic drugs may also be applied to the anchor and released therefrom over time.

The present inventions also include any and all combinations of the elements from the various embodiments disclosed in the specification, and systems that comprise sources of heated fluid and/or current for resistive heating in combination with any of the device described above and/or claimed below. It is intended that the scope of the present inventions extend to all such modifications and/or additions and that the scope of the present inventions is limited solely by the claims set forth below.

I claim:

1. A prosthetic valve, comprising:
an anchor formed from a shape memory material having martensitic state, an austenitic state, and a transition condition that is not a normal body condition, the anchor defining a delivery size, being mechanically deformable to a deployment size which is larger than the delivery size when in the martensitic state, and trained to shrink to a repositioning size which is smaller than deployment size when exposed to the transition condition and to remain at the repositioning size when subsequently exposed to normal body conditions; and
a leaflet device carried by the anchor and movable between an open orientation and a closed orientation.

2. A prosthetic valve as claimed in claim 1, wherein the shape memory material comprises a thermally responsive shape memory material and the transition condition comprises a temperature above normal body temperature.

3. A prosthetic valve as claimed in claim 1, wherein the shape memory material comprises a ferromagnetic shape memory material and the transition condition comprises a magnetic field.

4. A prosthetic valve as claimed in claim 1, wherein the anchor defines a generally tubular shape.

5. A prosthetic valve as claimed in claim 1, wherein the anchor comprises a plurality of wires.

6. A prosthetic valve as claimed in claim 1, wherein the anchor comprises a tube with a plurality of apertures.

7. A prosthetic valve as claimed in claim 1, wherein the leaflet device comprises a plurality of leaflets.

8. A prosthetic valve as claimed in claim 1, wherein the leaflet device is formed from at least one of natural tissue and synthetic material.

9. A prosthetic valve as claimed in claim 1, further comprising:
at least one protrusion extending outwardly from the anchor.

10. A prosthetic valve as claimed in claim 1, wherein the delivery size is a size suitable for endovascular delivery.

11. A prosthetic valve as claimed in claim 1, wherein the repositioning size is a size suitable for repositioning within the aortic valve region of the heart.

12. A method for use with a prosthetic valve having a leaflet device and a valve anchor with a martensitic state and an austenitic state, the method comprising the step of:
shrinking the valve anchor from a deployed size to a smaller repositioning size by causing the valve anchor to transition from the martensitic state to the austenitic state.

13. A method as claimed in claim 12, wherein the step of shrinking the valve anchor comprises shrinking the valve anchor from a deployed size to a smaller repositioning size by imparting a transition condition onto the valve anchor that causes the valve anchor to transition from the martensitic state to the austenitic state.

14. A method as claimed in claim 12, wherein the step of shrinking the valve anchor comprises shrinking the valve anchor from a deployed size to a smaller repositioning size by heating the valve anchor to a transition temperature that is above body temperature and causes the valve anchor to transition from the martensitic state to the austenitic state.

15. A method as claimed in claim 12, wherein the step of shrinking the valve anchor comprises shrinking the valve anchor from a deployed size to a smaller repositioning size by applying a magnetic field to the valve anchor that causes the valve anchor to transition from the martensitic state to the austenitic state.

16. A method as claimed in claim 12, further comprising the step of:
moving the valve after the valve anchor has returned to the martensitic state.

17. A method as claimed in claim 16, further comprising the step of:
redeploying the valve, by expanding and mechanically deforming the valve anchor while the valve anchor is in the martensitic state, after moving the valve.

18. A method as claimed in claim 12, further comprising the step of:
deploying the valve, by expanding and mechanically deforming the valve anchor while the valve anchor is in the martensitic state, prior to shrinking the valve anchor.

19. A method as claimed in claim 12, wherein the step of shrinking the valve anchor comprises shrinking the valve anchor, from a deployed size to a smaller repositioning size, onto an expandable device by imparting a transition condition onto the valve anchor that causes the valve anchor to transition from the martensitic state to the austenitic state.

20. A method as claimed in claim 19, further comprising the step of:
redeploying the valve by expanding and mechanically deforming the valve anchor with the expandable device.

21. An assembly, comprising:
a probe defining a longitudinal axis;
an expandable device, carried by the probe, including distal portion and a carrying portion, the distal portion defining a larger cross-sectional size perpendicular to the longitudinal axis than the carrying portion; and
a prosthetic valve including a leaflet device and an anchor formed from shape memory material carried by the carrying portion of the expandable device.

22. An assembly as claimed in claim 21, wherein the probe comprises a catheter body.

23. An assembly as claimed in claim 21, wherein the expandable device comprises an inflatable device.

24. An assembly as claimed in claim 21, wherein the anchor is crimped on the expandable device.

25. An assembly as claimed in claim 21, wherein the carrying portion comprises the distal portion of the expandable device.

26. An assembly as claimed in claim 21, wherein
the expandable device includes a proximal portion defining a larger cross-sectional size perpendicular to the longitudinal axis than the carrying portion; and
the carrying portion is located between the distal portion and the proximal portion.

27. A prosthetic valve, comprising:
an anchor including at least one structural member formed from a shape memory material and defining a non-constant cross-sectional size; and
a leaflet device carried by the anchor and movable between an open orientation and a closed orientation.

28. A prosthetic valve as claimed in claim 27, wherein the at least one structural member comprises a wire with at least a first portion having a first cross-sectional size and a second portion having a second cross-sectional size that is less than the first cross-sectional size.

29. A prosthetic valve as claimed in claim 28, wherein the first portion defines a first cross-sectional size shape and the second portion defines a second cross-sectional size that is different than the first cross-sectional shape.

30. A prosthetic valve as claimed in claim 27, wherein the at least one structural member comprises a tube with at least a first portion having a first wall thickness and a second portion having a second wall-thickness that is less than the first wall thickness.

31. A prosthetic valve as claimed in claim 30, wherein the tube defines an outer diameter and the outer diameter of the tube is substantially constant.

32. A prosthetic valve as claimed in claim 30, wherein the anchor defines an outer diameter and the outer diameter of the anchor is non-constant.
